(19) United States
(12) Patent Application Publication
(10) Pub. No.: US 2006/0100474 A1
(43) Pub. Date: May 11, 2006

(54) Method of making mixtures of internal olefins

(75) Inventor: David A. Capwell, West Greenwich, RI (US)

Correspondence Address: EDWARDS & ANGELL, LLP, P.O. BOX 55874, BOSTON, MA, 02205, US

(73) Assignee: Bercen Incorporated

(21) Appl. No.: 11/271,010

(22) Filed: Nov. 10, 2005

Related U.S. Application Data
(60) Provisional application No. 60/626,847, filed on Nov. 10, 2004.

(51) Int. Cl. C07C 5/25

(52) U.S. Cl. 585/670

(57) ABSTRACT

The invention provides new methods for the synthesis of isomeric mixtures of alkenes from α-olefins or mixtures of internal and terminal alkenes (i.e., predominantly α-olefins). The invention describes the process for producing an isomeric mixture of at least one internal alkene comprising contacting at least one or a mixture of alkene feedstock with a heterogeneous catalyst comprising a group IV metal oxide at a temperature and pressure conducive to positional isomerization of the double bond. The methods of the invention are particularly suitable for the preparation of isomeric mixtures of olefins suitable for use as additives in the paper making process and particularly as ASA sizing agents.

### RELATED APPLICATION

This application claims priority to U.S. Provisional Patent Application Ser. No. 60/626,847, filed Nov. 10, 2004, the disclosure of which is incorporated herein in its entirety by this reference.

### BACKGROUND

1. Field of the Invention

The invention provides new processes for producing isomeric mixtures of internal olefins by isomerization of a corresponding α-olefin (terminal olefin) or mixtures of olefins comprising at least one α-olefin and one or more internal isomers thereof (β, 67 , etc.), in the presence of heterogeneous titania or supported titania catalysts. The invention further provides processes for isomerizing olefins or olefin blends ranging from 8 to 40 carbon atoms, preferably around 12 to 24 carbon atoms.

2. Background of the Invention

The isomerization of olefins can be catalyzed by a variety of agents including acids, bases, and a number of transition metal complexes. These catalysts yield a thermodynamic mixture of isomeric olefins if the isomerizations are allowed to proceed to equilibrium. Non-thermodynamic distributions of products are obtained if equilibrium is not achieved, but it is rare that olefin isomerization leads to high yields of one isomer unless it is highly favored thermodynamically.

Numerous catalysts and synthetic procedures have been developed to isomerize olefins. Thus a number of catalysts and reaction conditions have been developed to reposition the carbon-carbon double bond in an alkene molecule. More particularly, catalytic systems which convert terminal olefins to internal olefins and catalytic systems which convert internal olefins to terminal olefins (e.g., α-olefins) have been developed.

Catalysts which have been employed for the positional isomerization of alkene C═C double bonds include, but are not limited to acids, ion-exchange materials, heterogeneous metal catalysts and solution phase metal complexes. Unfortunately, the catalytic systems developed to date have been hampered by one or more undesirable traits, such as, but not limited to expensive reagents, complex product mixtures, impurities or byproducts, low catalyst lifetime, harsh reaction conditions or a combination thereof.

Inexpensive reactions for the conversion of olefin precursors such as α-olefin feed stocks to deeply internalized alkenes, e.g., alkenes in which the C═C double bond is remote from the terminus of the carbon backbone of the olefin, have been challenging to develop.

Although there have been reports in the literature for the synthesis of deeply internalized olefins starting from α-olefins, these synthetic routes require multiple reaction steps, often provide undesirable products, and often require costly catalysts.

Thus, it would be desirable to provide new processes to synthesize deeply internalized olefins in a single step from an inexpensive α-olefin or alkene mixture. It would be particularly desirable to have synthetic procedures for the preparation of linear internal alkenes, which require a single process step and utilize inexpensive and chemically robust catalysts.

### SUMMARY OF THE INVENTION

In one aspect, the instant invention provides a process for producing an isomeric mixture of at least one x-Cₙ-alkene comprising contacting at least one Cₙ-alkene feedstock with a heterogeneous catalyst system comprising a group IV metal oxide at a temperature and pressure conducive to positional isomerization of the double bond of Cₙ-alkene feedstock, and recovering said isomeric mixture of at least one x-Cₙ-alkene, wherein n is one or more integers selected from 8 to 40; and x defines the position of the alkene double bond in the n-carbon alkene chain wherein the average value of x is between 2 and n/2. In one embodiment, the average value of x is between about 3 and n/2.

In another embodiment, the Cₙ-alkene feedstock is one or more α-alkenes, one or more linear alkenes having a disubstituted double bond, or a combination thereof. In certain embodiments, the Cₙ-alkene feedstock is one or more linear alkenes having a disubstituted double bond.

In another embodiment, the heterogeneous catalyst system comprises between 0.1% and about 100% titanium oxide by weight. Preferably, the heterogeneous catalyst system comprises between 50% and about 100% titanium oxide by weight. More preferably, the heterogeneous catalyst system comprises between 75% and about 100% titanium oxide by weight.

In still another embodiment, the heterogeneous catalyst system comprises titanium oxide and at least one additional material selected from Rh, Ir, Ni, Pd. Pt, and solid acid catalysts. In a further embodiment, the additional catalyst is a Pd catalyst. In a further embodiment, the additional material selected from Rh, Ir, Ni, Pd, Pt, and solid acid catalysts initially converts the Cₙ-alkene feedstock to an isomeric mixture of at least one z-Cₙ-alkene, wherein n is one or more integers selected from 8 to 40; and z defines the position of the alkene double bond in the n-carbon alkene chain wherein the average value of z is between 2 and n/2. Preferably, the average value of z is between about 3 and less than n/2. In a further embodiment, the additional material is a Pd catalyst. In a further embodiment, the heterogeneous catalyst system comprising a group IV metal oxide isomerizes the double bond of the z-Cₙ-alkene to a mixture of x-Cₙ-alkene, wherein n is one or more integers selected from 8 to 40; and x defines the position of the alkene double bond in the n-carbon alkene chain wherein the average value of x is between 2 and n/2 or more preferably greater than 3 and less than n/2; and wherein x is greater than z.

In other embodiments, the heterogeneous catalyst system comprises a group IV metal oxide and at least one other solid capable of providing structural support. In yet another embodiment, the group IV metal oxide is titanium oxide and the solid capable of providing structural support is selected from silica, alumina, carbon, diatomaceous clays, and mixtures thereof. In a further embodiment, the heterogeneous support comprises between about 25% and about 95% titanium oxide and between about 75% and about 5% of a structural material selected from silica, alumina, or carbon, diatomaceous clays, and mixtures thereof. In other embodiments, the heterogeneous metal oxide comprises between about 0.1% and about 20% of a sulfate salt or sulfuric acid by weight of the heterogeneous metal oxide. Preferably, the heterogeneous metal oxide comprises between about 1% and about 10% of a sulfate salt or sulfuric acid by weight of the heterogeneous metal oxide. More preferably, the heterogeneous metal oxide comprises between about 3% and about 7% of a sulfate salt or sulfuric acid by weight of the heterogeneous metal oxide.

In one embodiment, the heterogeneous catalyst has a surface area of between about 50 and about 500 m²/g. In a further embodiment, the heterogeneous catalyst has a surface area of between about 150 and about 300 m²/g.

In another embodiment, the heterogeneous catalyst has a pore diameter of between about 50 Å and about 400 Å. Preferably, the heterogeneous catalyst has a pore diameter of between about 100 Å and about 200 Å.

In certain embodiments, the Cₙ-alkene feedstock is selected from α-alkenes having between 12 and 30 carbon atoms, internal alkenes having between 12 and 30 carbon atoms, and mixtures thereof. In a further embodiment, the Cₙ-alkene feedstock is selected from 1-dodecene, 1-tetradecene, 1-hexadecene, 1-octadecene, 1-eicosene, 1-docosene, 1-tetracosene, or a mixture thereof. In another further embodiment, the Cₙ-alkene feedstock is selected from 1-tetradecene, 1-hexadecene, 1-octadecene, 1-eicosene, or a mixture thereof. In some embodiments, the Cₙ-alkene feedstock is a mixture of 1-hexadecene and 1-octadecene. In another further embodiment, the ratio of 1-hexadecene to 1-octadecene is between about 1:10 and about 10:1. In certain embodiments, the Cₙ-alkene feedstock consists essentially of 1-hexadecene or 1-octadecene.

In one embodiment, the feedstock is selected from 1-dodecene, 1-tetradecene, 1-hexadecene, 1-octadecene, 1-eicosene, 1-docosene, 1-tetracosene, and internal alkenes wherein n is 12-30; and mixtures thereof. In another embodiment, the feedstock is selected from internal alkenes wherein n is 12-30.

In another embodiment, the Cₙ-alkene feedstock is contacted with the heterogeneous catalyst at a temperature of about 500° C. or less. Preferably, the Cₙ-alkene feedstock is contacted with the heterogeneous catalyst at a temperature of between about 100° C. and about 400° C. In certain embodiments, the Cₙ-alkene feedstock is contacted with the heterogeneous catalyst at a pressure of less than about 100 atmospheres. Preferably, the Cₙ-alkene feedstock is contacted with the heterogeneous catalyst at a pressure of between about 1 and about 75 atmospheres. In other embodiments, the Cₙ-alkene feedstock is contacted with the heterogeneous catalyst in the absence of water.

In another embodiment, the isomeric mixture of x-Cₙ-alkenes is selected from alkene mixtures in which the average value of x is between about 3 and about 7, and n is one, two, three, or four integers of between 12 and 24. In a further embodiment, at least about 40% of the occurrences of x is an integer of between 3 and about n/2. In another further embodiment, at least about 80% of the occurrences of x is an integer of between 3 and about n/2.

In another embodiment, the isomeric mixture of at least one x-Cₙ-alkene comprises at least about 40% of gamma, delta, epsilon, and eta-alkene positional isomers of the x-Cₙ-alkene mixture. In a further embodiment, the isomeric mixture of at least one x-Cₙ-alkene comprises at least about 80% of gamma, delta, epsilon, and eta alkene positional isomers of the x-Cₙ-alkene mixture. In another further embodiment, the Cₙ-alkene is tetradecene, hexadecene, octadecene, eicosene, or a mixture thereof. In another embodiment the Cₙ-alkene is hexadecene, octadecene, or a mixture thereof.

In still another embodiment, the isomeric mixture of x-Cₙ-alkenes is selected from alkene mixtures in which the average value of x is between about 2 and about 7, and n is one, two or three integers of between 12 and 24. In certain embodiments, at least about 40% of the occurrences of x is an integer of between 1 and about 7. In a further embodiment, at least about 80% of the occurrences of x is an integer of between 2 and about 6.

In another embodiment, the isomeric mixture of at least one x-Cₙ-alkene comprises at least about 40% of alpha, beta, gamma, delta, epsilon, and eta-alkene positional isomers of the x-Cₙ-alkene mixture. Preferably, the isomeric mixture of at least one x-Cₙ-alkene comprises at least about 80% of beta, gamma, delta, epsilon, and eta alkene positional isomers of the x-Cₙ-alkene mixture. In a further embodiment, the Cₙ-alkene is tetradecene, hexadecene, octadecene, eicosene, or a mixture thereof. In a further embodiment, the Cₙ-alkene is hexadecene, octadecene, or a mixture thereof.

In yet another embodiment, the Cₙ-alkene feedstock is contacted with the heterogeneous catalyst system in a continuous flow, fixed bed reactor. In another embodiment, the Cₙ-alkene feedstock is contacted with the heterogeneous catalyst system in a batch reactor. In a further embodiment, a flow of Cₙ-alkene feedstock is contacted with the heterogeneous catalyst system at a rate of between about 0.1 to about 50 (kilograms Cₙ-alkene feedstock per hour per kilogram of heterogeneous catalyst). In still another embodiment, the catalyst can act as a stand alone catalyst, a pre-isomerization catalyst, or a tailing catalyst. In certain embodiments, the catalyst is a tailing catalyst.

In another aspect, the invention provides a process for producing an isomeric mixture of at least one x-Cₙ-alkene comprising contacting at least one z-Cₙ-alkene feedstock with a heterogeneous catalyst system comprising a group IV metal oxide at a temperature and pressure conducive to positional isomerization of the double bond of Cₙ-alkene feedstock, and recovering said isomeric mixture of at least one x-Cₙ-alkene, wherein n is one or more integers selected from 8 to 40; x defines the position of the alkene double bond in the n-carbon alkene chain wherein the average value of x is between 2 and n/2; z defines the position of the alkene double bond in the Cₙ-alkene feedstock, wherein the average value of z is between 1 and n/2; and the average value of n is greater than the average value of z. Preferably, the average value of x is between about 3 and n/2.

In one embodiment, the Cₙ-alkene feedstock is one or more α-alkenes, one or more linear alkenes having a disubstituted double bond, or a combination thereof. In another embodiment, the Cₙ-alkene feedstock is one or more linear alkenes having a disubstituted double bond.

In another aspect, the invention provides a process for producing an isomeric mixture of at least one x-Cₙ-alkene comprising,

a) contacting the Cₙ-alkene feedstock with an additional material selected from Rh, Ir, Ni, Pd, Pt, and solid acid catalysts to initially convert the Cₙ-alkene feedstock to an isomeric mixture of at least one z-Cₙ-alkene, wherein n is one or more integers selected from 8 to 40; and z defines the position of the alkene double bond in the n-carbon alkene chain wherein the average value of z is between 2 and n/2;

b) contacting the z-Cₙ-alkene with a heterogeneous catalyst system comprising a group IV metal oxide at a temperature and pressure conducive to positional isomerization of the double bond and recovering an isomeric mixture of at least one x-Cₙ-alkene;

wherein n is one or more integers selected from 8 to 40;

x defines the position of the alkene double bond in the n-carbon alkene chain wherein the average value of x is between 2 and n/2;

z defines the position of the alkene double bond in the Cₙ-alkene feedstock;

the average value of z is between 1 and n/2; and

the average value of x is greater than the average value of z.

### DETAILED DESCRIPTION OF THE INVENTION

We have discovered new methods for the preparation of deeply internalized olefins, including mixtures of positional isomers of x-Cₙ-alkenes in which at least 40% of the alkene double bond is in the 3-position or a more highly internalized position. The invention describes the process for producing an isomeric mixture of at least one x-Cₙ-alkene comprising contacting at least one Cₙ-alkene feedstock with a heterogeneous catalyst comprising a group IV metal oxide at a temperature and pressure conducive to positional isomerization of the double bond of Cₙ-alkene feedstock, and recovering said isomeric mixture of at least one x-Cₙ-alkene, wherein n is one or more integers selected from 8 to 40; and x defines the position of the alkene double bond in the n-carbon alkene chain wherein the average value of x is between 2 and n/2 or more preferably between about 3 and n/2.

The internal olefins are typically used for alkenyl succinic anhydride (ASA) sizing agents in the alkaline production of paper. Sizing agents can be used as additives for decreasing the penetration or spreading of aqueous solutions through or across paper. In a typically preferred method of synthesis, ASA sizing agents are prepared by contacting maleic anhydride with an internal olefin.

Group IV metals are known to persons skilled in the art and include metals of Group IV of the Periodic Table of the Elements. Examples of suitable metals include Group IV metals Ti, Zr, and Hf. The Group IV metal in the above mentioned catalyst is preferably Ti or a mixture of Ti with Zr and/or Hf, or a mixture of Ti with one or more additional transition metals. Typically preferred catalysts include Ti, Ti/Zr alloys, metal oxides of Ti, and mixed metal oxides of Ti and Zr, each of which may option have trace Hf impurities in the catalyst. The metal component may be present as an oxide state in the catalyst product.

In one aspect of the invention, the heterogeneous catalyst comprises between 0.1% and about 100% titanium oxide by weight. In a preferred embodiment, the heterogeneous catalyst comprises between 50% and about 100% titanium oxide by weight. In a preferred embodiment, the heterogeneous catalyst comprises between 75% and about 100% titanium oxide by weight. In another embodiment, the catalyst according to the present invention can also contain other components or mixtures thereof, which act alone or are combined as catalyst modifiers to improve the activity, selectivity or stability of the catalyst. In certain embodiments, the methods comprise a heterogeneous catalyst, which catalyst comprises titanium oxide and at least one additional material selected from Rh, Ir, Ni, Pd, Pt, and solid acid catalysts.

In another embodiment, the heterogeneous catalyst comprises a group IV metal oxide and at least one other solid capable of providing structural support. A preferred embodiment comprises titanium oxide as the group IV metal oxide and the solid capable of providing structural support is selected from silica, alumina, carbon, diatomaceous clays, and mixtures thereof. In accordance with another embodiment in this invention, the heterogeneous support comprises between about 25% and about 95% titanium oxide and between about 75% and about 5% of a structural material selected from silica, alumina, or carbon, diatomaceous clays, and mixtures thereof.

The catalyst support is generally a porous adsorptive material. In certain embodiments, the porous support has a uniform composition, used herein to mean that the support is not layered and has no concentration gradient of the intrinsic components. If the support is a mixture of two or more materials, these materials have a relative constant content or a uniform distribution throughout the whole support. Typical catalyst supports include inorganic materials, such as alumina, magnesia, chromia, boron oxide, titania, thoria, zinc oxide, zirconia, or the mixtures thereof, various ceramics, various alumine, and various bauxites; silica, silicon carbide, various synthetic or natural silicates and clays (including natural and synthetic zeolites). These silicates and clays may be untreated or treated with one or more acids.

In another embodiment, the heterogeneous metal oxide comprises between about 0.1% and about 20% of a sulfate salt or sulfuric acid by weight of the heterogeneous metal oxide. In one preferred embodiment of the invention, the heterogeneous metal oxide comprises between about 1% and about 10% of a sulfate salt or sulfuric acid by weight of the heterogeneous metal oxide. In another preferred embodiment of the invention, the heterogeneous metal oxide comprises between about 3% and about 7% of a sulfate salt or sulfuric acid by weight of the heterogeneous metal oxide. The resulting catalyst is a sulfate doped group IV metal oxide catalyst. Sulfate salts include those groups having one or more sulfate (SO₄²⁻) or bisulfate (HSO₄¹⁻) anions.

In another embodiment of the invention, the heterogeneous catalyst has a surface area of between about 50 and about 500 m²/g. In certain preferred embodiments, the heterogeneous catalyst has a surface area of between about 150 and about 300 m²/g. In certain other preferred embodiments, the heterogeneous catalyst has a surface area of about 100 m²/g, about 150 m²/g, about 200 m²/g, about 250 m²/g, or about 300 m²/g.

In another embodiment of the invention, the heterogeneous catalyst has a pore diameter of between about 50 Å and about 400 Å. In certain preferred embodiments, the heterogeneous catalyst has a pore diameter of between about 100 Å and about 200 Å. In certain preferred embodiments, the heterogeneous catalyst has an average pore diameter of between about 50-100 Å, between about 100-150 Å, between about 150-200 Å, between about 200-250 Å, between about 250-300 Å, or between about 50-150 Å, between about 150-250 Å, and between about 200-300 Å.

An example of a preferred catalyst is the DeGussa Aerolyst 7752. This titania (TiO₂) and silica catalyst is based on fumed oxides for fixed bed applications. Aerolyst 7752 has been reported by the manufacturer as a catalyst for reactions of organic substrates with water. In contrast, we have surprisingly discovered that Aerolyst 7752 or other Aerolyst 77xx series products function as heterogenous solid acid catalysts for double bond isomerization of alkenes when the reaction is conducted substantially in the absence of water. The Aerolyst 7752 product line satisfies the parameters set forth in Table 1.

TABLE 1
General physical properties of the Degussa Aerolyst 7752 sulfate
doped titania

| | |
|---|---|
| Titania (TiO₂) | 100% |
| Surface SO₄ | about 4-6% |
| Macroscale morphology | Any (preferably an extrudate such as a ring, |
| | star, tablet, etc.) |
| size: | typically about 4-5 mm |
| surface area | 170-200 m²/g |
| pore volume | 0.3-0.4 ml/g |
| pore diameter | 120-170 Å |
| working temperature | 100° C.-500° C. (175° C.) |
| working pressure | 10-1000 psi |

As used herein, the term “alkene” refers to organic molecules having at least two carbon atoms and at least one carbon-carbon double bond. The term “olefin” is used interchangeably with the term “alkene”. The instant invention provides methods of isomerizing alkenes of carbon chains of at least four carbons in length, as required for olefin isomerization. In certain embodiments additional elements may be present in the alkene feed stock, which elements are typically selected from oxygen, nitrogen, sulfur, fluorine, chlorine, and bromine. In typically preferred methods of the invention the alkene feed stock is a mono-unsaturated hydrocarbon which consists essentially of carbon and hydrogen.

Typically preferred alkenes include linear or branched alkenes having a longest carbon backbone of at least 5 carbon atoms, preferably at least 8 carbon atoms, which carbon backbone typically comprises at least one terminal or internal carbon-carbon double bond. In general, alkene feedstocks are referred to herein based on the number of carbons in the alkene molecule, e.g., a C₁₂-alkene is dodecene, C14-alkene is tetradecene, hexadecene is a C₁₆-alkene, and octadecene is a C₁₈-alkene. Typically, the alkene feedstock is selected from α-alkenes having between 12 and 30 carbon atoms, internal alkenes having between 12 and 30 carbon atoms, and mixtures thereof.

Many geometric isomers of olefins, can be present in the compounds described herein, and all such stable isomers are contemplated in the present invention. Cis and trans geometric isomers of the compounds of the present invention are described and may be isolated as a mixture of isomers or as separated isomeric forms.

The alkenes herein described may have one or more asymmetric centers or planes. Compounds of the present invention containing an asymmetrically substituted atom may be isolated in optically active or racemic forms. It is well known in the art how to prepare optically active forms, such as by resolution of racemic forms (racemates), by asymmetric synthesis, or by synthesis from optically active starting materials. All chiral (enantiomeric and diastereomeric), and racemic forms, as well as all geometric isomeric forms of a structure are intended, unless the specific stereochemistry or isomeric form is specifically indicated.

Various substitution of the olefins means that any one or more hydrogens on the designated atom or group is replaced with a selection from the indicated group of substituents, provided that the designated atom's normal valence is not exceeded, and that the substitution results in a stable compound. When a substituent is oxo (keto, i.e., =O), then 2 hydrogens on an atom are replaced. The present invention is intended to include all isotopes (including radioisotopes) of atoms occurring in the present compounds. As to any of the above groups that contain one or more substituents, it is understood by those skilled in the art, that such groups do not contain any substitution or substitution patterns which are sterically unfeasible and or synthetically impracticable.

The Cₙ-alkene feedstock typically comprises an alkene with a double bond at the terminal position, an internal position, or a mixture thereof. In certain preferred embodiments, the Cₙ-alkene feedstock is selected from 1-dodecene, 1-tetradecene, 1-hexadecene, 1-octadecene, 1-eicosene, 1-docosene, 1-tetracosene, or a mixture thereof In another preferred embodiment, the Cₙ-alkene feedstock is selected from 1-tetradecene, 1-hexadecene, 1-octadecene, 1-eicosene, or a mixture thereof. In a further preferred embodiment, the Cₙ-alkene feedstock is a mixture of 1-hexadecene and 1-octadecene, wherein the ratio of 1-hexadecene to 1-octadecene is between about 1:10 and about 10:1, or wherein the Cₙ-alkene feedstock consists essentially of 1-hexadecene or 1-octadecene. In certain other embodiments, a Cₙ-alkene in which n is an odd number are also suitable for use in the isomerization methods of the invention.

The invention comprises reaction conditions used in the process of producing isomeric mixtures of internal olefins in the presence of group IV metal oxide catalysts. In a preferred embodiment of the invention, reaction conditions are maintained wherein the Cₙ-alkene feedstock is contacted with the heterogeneous catalyst at a temperature of about 500° C. or less, In a preferred embodiment, Cₙ-alkene feedstock is contacted with the heterogeneous catalyst at a temperature of between about 25° C. and about 400° C. In certain preferred embodiments, the reaction temperature is between about 50-500° C., or more preferably between about 100-250° C., between about 125-250° C., or between about 50-100° C., between about 100-150° C., between about 125-150° C., between about 150-175° C. or between about 175-200° C.

Another embodiment comprises reaction conditions wherein the Cₙ-alkene feedstock is contacted with the heterogeneous catalyst at a pressure of less than about 100 atmospheres. In a preferred embodiment, the method comprises contacting the Cₙ-alkene feedstock with the heterogeneous catalyst at a pressure of between about 1 and about 75 atmospheres, preferably between about 2 and about 75 atm. In certain other preferred methods of the invention, the Cₙ-alkene feedstock is contacted with the heterogeneous catalyst at a pressure of about 1 atm, about 2 atm, about 5 atm, about 10 atm, about 15 atm, about 20 atm, about 25 atm, about 30 atm, about 35 atm, about 40 atm, about 50 atm, about 60 atm, about 70 atm, or about 75 atm.

Another embodiment consists of reaction conditions wherein the Cₙ-alkene feedstock is contacted with the heterogeneous catalyst in the absence of water. The process must be carried out under a substantially water-free environment. The water content in the feedstock entering into the reactor should be less than 50 ppm, preferably less than 20 ppm, preferably less than 10 ppm. The water in the reforming feedstock may be removed by using the conventional adsorbents such as molecular sieves, copper sulfate, calcium chloride or other water scavenging reagents. Moisture can also be removed by the using a column filled with a high surface area packing and a concurrent ultra dry gas such as nitrogen.

Another aspect of the present invention comprises a process for producing an internal olefin or a mixture of internal olefin of different carbon number, as the isomeric mixture of x-Cₙ-alkenes is selected from alkene mixtures in which the average value of x is between about 3 and about 7, and n is one, two, three or four integers of between 12 and 24. In a preferred embodiment, at least about 40% of the occurrences of x is an integer of between 3 and about n/2. In another preferred embodiment, at least about 80% of the occurrences of x is an integer of between 3 and about n/2.

In another aspect of the invention, the process of producing internal olefins comprises the isomeric mixture of at least one x-Cₙ-alkene comprising at least about 40% of gamma, delta, epsilon, and eta-alkene positional isomers of the x-Cₙ-alkene mixture. Particularly preferred are at least about 80% of gamma, delta, epsilon, and eta alkene positional isomers of the x-Cₙ-alkene mixture.

A preferred embodiment comprises internal olefins wherein the Cₙ-alkene is tetradecene, hexadecene, octadecene, eicosene, or a mixture thereof. Another preferred embodiment comprises internal olefins wherein the Cₙ-alkene is hexadecene, octadecene, or a mixture thereof.

The terms gamma, delta, epsilon, and eta refer to positions along the carbon chain where the alkene begins. In this invention, gamma refers to the third carbon position along the carbon chain, delta refers to the fourth position, epsilon refers to the fifth position, and eta refers to the sixth position. As an example, a linear carbon chain of 12 carbons, substituted at the delta position by an olefin, is referred to as 4-dodecene.

In another aspect of the invention, the process of producing internal olefins comprises the isomeric mixture of at least one x-Cₙ-alkene wherein the isomeric mixture of x-Cₙ-alkenes is selected from alkene mixtures in which the average value of x is between about 2 and about 7, and n is one, two or three integers of between 12 and 24.

In another aspect, the invention provides a process of producing internal olefins comprises the isomeric mixture of at least one x-Cₙ-alkene wherein at least about 40% of the occurrences of x is an integer of between 1 and about 7. In a further preferred embodiment of the invention, at least about 80% of the occurrences of x is an integer of between 2 and about 6.

In another aspect of the invention, the process of producing internal olefins comprises the isomeric mixture of at least one x-Cₙ-alkene comprises at least about 40% of alpha, beta, gamma, delta, epsilon, and eta-alkene positional isomers of the x-Cₙ-alkene mixture. Particularly preferred are at least about 80% of beta, gamma, delta, epsilon, and eta alkene positional isomers of the x-Cₙ-alkene mixture.

A preferred embodiment comprises internal olefins wherein the Cₙ-alkene is tetradecene, hexadecene, octadecene, eicosene, or a mixture thereof. Another preferred embodiment comprises internal olefins wherein the Cₙ-alkene is hexadecene, octadecene, or a mixture thereof.

Although tetradecene, hexadecene, octadecene, eicosene, and mixtures thereof are preferred for use in preparation of feedstocks for ASA additives for paper making processes, other Cₙ-alkenes may be preferable for use in the preparation of other deeply internalized olefins which are suitable for use in a variety of other applications, such as lubricants or inexpensive feedstocks in the synthesis of lubricants. Preferably, the deeply internalized olefins can be used in the fabrication of functional fluids, such as drill fluids used in drilling mud. Advantageous properties of these fluids include desirable viscosity properties and biodegradability properties. Thus, in certain embodiments, the methods of the invention are suitable for preparing a broad distribution of positional alkene isomers in which the product mixture comprises α-olefin, β-olefin, and internal olefins where the double bond is at the third carbon or more deeply internalized.

In yet another aspect, the invention comprises contacting the Cₙ-alkene feedstock with the heterogeneous catalyst in a continuous flow, fixed bed reactor. In a preferred embodiment, the flow of Cₙ-alkene feedstock is contacted with the heterogeneous catalyst at a rate of between about 0.1 to about 50 (kilograms Cₙ-alkene feedstock per hour per kilogram of heterogeneous catalyst). In certain embodiments, the Cₙ-alkene feedstock is contacted with the a Pd catalyst in a continuous flow fixed bed reactor and then contacted with a second continuous flow fixed bed reactor with the heterogeneous catalyst system comprising a group IV metal oxide.

By “continuous flow reactor” as used herein we mean a reactor in which reactants are introduced and mixed and products withdrawn simultaneously in a continuous manner, as opposed to a batch process. For example, the reactor may be a plug flow reactor, although the various aspects of the invention defined herein are not limited to this particular type of continuous flow reactor.

In yet another aspect, the invention comprises contacting the Cₙ-alkene feedstock with the heterogeneous catalyst in a batch reactor.

By “batch reactor” as used herein we mean a reactor whereby the reactants and catalyst are placed in a reactor which is then closed to transport of matter and the reaction is allowed to proceed for a given time whereupon the mixture is withdrawn.

By “heterogeneous catalyst system” as used herein, we mean a catalyst system comprising a plurality of catalysts, wherein at least one catalyst in the catalyst system is a heterogeneous catalyst.

Contact of the various streams may be effected by way of separate feeds to a device in which the feeds are united to form a single homogeneous fluid phase. The device in which the feeds are united may for instance have a Y, T, X or other configuration allowing separate feeds to be united in a single flow passage forming the continuous flow reactor, or in some circumstances multiple flow passages forming two or more continuous flow reactors. The flow passage or passages in which the feeds are united may comprise a section of tubular configuration with or without internal dynamic or static mixing elements.

As an example, a continuous flow reactor can comprise a column of known diameter and length, packed with a heterogenous catalyst, which may be mixed with any other solid that will hold particle shape; the catalyst packing having a known mass, shape, size, surface area, pore volume, and pore diameter. The reaction temperature, flow rate of alkene feedstock, and pressure adjusted to the column.

In another aspect, the catalyst of the present invention can be used as a stand alone catalyst, a pre-isomerization catalyst, or a tailing catalyst, or in any combination thereof.

The invention has been described in detail with reference to preferred embodiments thereof. However, it will be appreciated that those skilled in the art, upon consideration of the disclosure, may make modifications and improvements within the spirit and scope of the invention.

The following non-limiting example is illustrative of the invention. All documents mentioned herein are incorporated herein by reference.

### EXAMPLE 1

Isomerization of 1-hexadecene to an internalized hexadecene was conducted using a continuous flow fixed bed reactor with the parameters provided in Table 2. The inlet and outlet temperature of the column are not identical but the overall operation temperature of the column is about 250° F. The isomerization product mixture provided by the isomerization reaction conducted under the conditions of Table 1 are provided in Table 3.

TABLE 2

| | |
|---|---|
| Number of Columns | 2 |
| Column diameter | 5 cm |
| Column length | 2.44 meters |
| Degussa's Aerolyst 7752 TiO2 - 5% active | 9 kilograms/column. |
| acid content | |
| 1-hexadecene flow rate through the column | 0.11 pounds per minute |

TABLE 3

| | |
|---|---|
| α-hexadecene | 1.22% |
| β-hexadecene | 12.42% |
| γ-hexadecene | 10.77% |
| δ-hexadecene | 14.94% |
| Ε-hexadecene | 41.90% |
| ζ-hexadecene | 18.75% |

### EXAMPLE 2

The conditions provided in Example 1 can be modified to provide a mixture of alkene isomers which are suitable for use when deep internalization is not necessary. Thus, modification of the reaction conditions can produce a mixture of less than 20% 1-hexadecene, less than 40% 2-hexadecene and more than 40% of 3+hexadecene.

What is claimed is:

1. A process for producing an isomeric mixture of at least one x-Cₙ-alkene comprising contacting at least one Cₙ-alkene feedstock with a heterogeneous catalyst system comprising a group IV metal oxide at a temperature and pressure conducive to positional isomerization of the double bond of Cₙ-alkene feedstock, and recovering said isomeric mixture of at least one x-Cₙ-alkene, wherein n is one or more integers selected from 8 to 40; and x defines the position of the alkene double bond in the n-carbon alkene chain wherein the average value of x is between 2 and n/2.

2. The process of claim 1, wherein the average value of x is between about 3 and n/2.

3. The process of claim 1, wherein the Cₙ-alkene feedstock is one or more α-alkenes, one or more linear alkenes having a disubstituted double bond, or a combination thereof.

4. The process of claim 1, wherein the Cₙ-alkene feedstock is one or more linear alkenes having a disubstituted double bond.

5. The process of claim 1, wherein the heterogeneous catalyst system comprises between 0. 1% and about 100% titanium oxide by weight.

6. The process of claim 5, wherein the heterogeneous catalyst system comprises between 50% and about 100% titanium oxide by weight.

7. The process of claim 6, wherein the heterogeneous catalyst system comprises between 75% and about 100% titanium oxide by weight.

8. The process of claim 1, wherein the heterogeneous catalyst system comprises titanium oxide and at least one additional material selected from Rh, Ir, Ni, Pd, Pt, and solid acid catalysts.

9. The process of claim 8, wherein the additional catalyst is a Pd catalyst.

10. The process of claim 1, wherein the heterogeneous catalyst system comprises a group IV metal oxide and at least one other solid capable of providing structural support.

11. The process of claim 10, wherein the group IV metal oxide is titanium oxide and the solid capable of providing structural support is selected from silica, alumina, carbon, diatomaceous clays, and mixtures thereof.

12. The process of claim 10, wherein the heterogeneous support comprises between about 25% and about 95% titanium oxide and between about 75% and about 5% of a structural material selected from silica, alumina, or carbon, diatomaceous clays, and mixtures thereof.

13. The process of claim 1, wherein the heterogeneous metal oxide comprises between about 0. 1% and about 20% of a sulfate salt or sulfuric acid by weight of the heterogeneous metal oxide.

14. The process of claim 13, wherein the heterogeneous metal oxide comprises between about 1% and about 10% of a sulfate salt or sulfuric acid by weight of the heterogeneous metal oxide.

15. The process of claim 14, wherein the heterogeneous metal oxide comprises between about 3% and about 7% of a sulfate salt or sulfuric acid by weight of the heterogeneous metal oxide.

16. The process of claim 1, wherein the heterogeneous catalyst has a surface area of between about 50 and about 500 m²/g.

17. The process of claim 16, wherein the heterogeneous catalyst has a surface area of between about 150 and about 300 m²/g.

18. The process of claim 1, wherein the heterogeneous catalyst has a pore diameter of between about 50 Å and about 400 Å.

19. The process of claim 18, wherein the heterogeneous catalyst has a pore diameter of between about 100 Å and about 200 Å.

20. The process of claim 1, wherein the Cₙ-alkene feedstock is selected from α-alkenes having between 12 and 30 carbon atoms, internal alkenes having between 12 and 30 carbon atoms, and mixtures thereof.

21. The process of claim 20, wherein the Cₙ-alkene feedstock is selected from 1-dodecene, 1-tetradecene, 1-hexadecene, 1-octadecene, 1-eicosene, 1-docosene, 1-tetracosene, or a mixture thereof.

22. The process of claim 20, wherein the Cₙ-alkene feedstock is selected from 1-tetradecene, 1-hexadecene, 1-octadecene, 1-eicosene, or a mixture thereof.

23. The process of claim 20, wherein the Cₙ-alkene feedstock is a mixture of 1-hexadecene and 1-octadecene.

24. The process of claim 23, wherein the ratio of 1-hexadecene to 1-octadecene is between about 1:10 and about 10:1.

25. The process of claim 20, wherein the Cₙ-alkene feedstock consists essentially of 1-hexadecene or 1-octadecene.

26. The process of claim 20, wherein the feedstock is selected from 1-dodecene, 1-tetradecene, 1-hexadecene, 1-octadecene, 1-eicosene, 1-docosene, 1-tetracosene, and internal alkenes wherein n is 12-30; and mixtures thereof.

27. The process of claim 20, wherein the feedstock is selected from internal alkenes wherein n is 12-30.

28. The process of claim 1, wherein the Cₙ-alkene feedstock is contacted with the heterogeneous catalyst at a temperature of about 500° F. or less.

29. The process of claim 28, wherein the Cₙ-alkene feedstock is contacted with the heterogeneous catalyst at a temperature of between about 250° F. and about 400° F.

30. The process of claim 1, wherein the Cₙ-alkene feedstock is contacted with the heterogeneous catalyst at a pressure of less than about 100 atmospheres.

31. The process of claim 30, wherein the Cₙ-alkene feedstock is contacted with the heterogeneous catalyst at a pressure of between about 1 and about 75 atmospheres.

32. The process of claim 1, wherein the Cₙ-alkene feedstock is contacted with the heterogeneous catalyst in the absence of water.

33. The process of claim 1, wherein the isomeric mixture of x-Cₙ-alkenes is selected from alkene mixtures in which the average value of x is between about 3 and about 7, and n is one, two, three, or four integers of between 12 and 24.

34. The process of claim 33, wherein at least about 40% of the occurrences of x is an integer of between 3 and about n/2.

35. The process of claim 34, wherein at least about 80% of the occurrences of x is an integer of between 3 and about n/2.

36. The process of claim 1, wherein the isomeric mixture of at least one x-Cₙ-alkene comprises at least about 40% of gamma, delta, epsilon, and eta-alkene positional isomers of the x-Cₙ-alkene mixture.

37. The process of claim 36, wherein the isomeric mixture of at least one x-Cₙ-alkene comprises at least about 80% of gamma, delta, epsilon, and eta alkene positional isomers of the x-Cₙ-alkene mixture.

38. The process of claim 36, wherein the Cₙ-alkene is tetradecene, hexadecene, octadecene, eicosene, or a mixture thereof.

39. The process of claim 36, wherein the Cₙ-alkene is hexadecene, octadecene, or a mixture thereof.

40. The process of claim 1, wherein the isomeric mixture of x-Cₙ-alkenes is selected from alkene mixtures in which the average value of x is between about 2 and about 7, and n is one, two or three integers of between 12 and 24.

41. The process of claim 1, wherein at least about 40% of the occurrences of x is an integer of between 1 and about 7.

42. The process of claim 41, wherein at least about 80% of the occurrences of x is an integer of between 2 and about 6.

43. The process of claim 1, wherein the isomeric mixture of at least one x-Cₙ-alkene comprises at least about 40% of alpha, beta, gamma, delta, epsilon, and eta-alkene positional isomers of the x-Cₙ-alkene mixture.

44. The process of claim 43 wherein the isomeric mixture of at least one x-Cₙ-alkene comprises at least about 80% of beta, gamma, delta, epsilon, and eta alkene positional isomers of the x-Cₙ-alkene mixture.

45. The process of claim 43, wherein the Cₙ-alkene is tetradecene, hexadecene, octadecene, eicosene, or a mixture thereof.

46. The process of claim 43, wherein the Cₙ-alkene is hexadecene, octadecene, or a mixture thereof.

47. The process of claim 8, wherein the additional material selected from Rh, Ir, Ni, Pd, Pt, and solid acid catalysts initially converts the Cₙ-alkene feedstock to an isomeric mixture of at least one z-Cₙ-alkene, wherein n is one or more integers selected from 8 to 40; and z defines the position of the alkene double bond in the n-carbon alkene chain wherein the average value of z is between 2 and n/2.

48. The process of claim 47, wherein the average value of z is between about 3 and less than n/2.

49. The process of claim 48, wherein the additional material is a Pd catalyst.

50. The process of claim 49, wherein the heterogeneous catalyst system comprising a group IV metal oxide isomerizes the double bond of the z-Cₙ-alkene to a mixture of x-Cₙ-alkene, wherein n is one or more integers selected from 8 to 40; and x defines the position of the alkene double bond in the n-carbon alkene chain wherein the average value of x is between 2 and n/2 or more preferably greater than 3 and less than n/2; and wherein x is greater than z.

51. The process of claim 1, wherein the Cₙ-alkene feedstock is contacted with the heterogeneous catalyst system in a continuous flow, fixed bed reactor.

52. The process of claim 1, wherein the Cₙ-alkene feedstock is contacted with the heterogeneous catalyst system in a batch reactor.

53. The process of claim 51, wherein a flow of Cₙ-alkene feedstock is contacted with the heterogeneous catalyst system at a rate of between about 0.1 to about 50 (kilograms Cₙ-alkene feedstock per hour per kilogram of heterogeneous catalyst).

54. The process of claim 1, wherein the catalyst can act as a stand alone catalyst, a pre-isomerization catalyst, or a tailing catalyst.

55. The process of claim 54, wherein the catalyst is a tailing catalyst.

56. A process for producing an isomeric mixture of at least one x-Cₙ-alkene comprising contacting at least one z-Cₙ-alkene feedstock with a heterogeneous catalyst system comprising a group IV metal oxide at a temperature and pressure conducive to positional isomerization of the double bond of Cₙ-alkene feedstock, and recovering said isomeric mixture of at least one x-Cₙ-alkene, wherein n is one or more integers selected from 8 to 40;
x defines the position of the alkene double bond in the n-carbon alkene chain wherein the average value of x is between 2 and n/2;
z defines the position of the alkene double bond in the Cₙ-alkene feedstock, wherein the average value of z is between 1 and n/2; and
the average value of x is greater than the average value of z.

57. The process of claim 56, wherein the average value of x is between about 3 and n/2.

58. The process of claim 56, wherein the Cₙ-alkene feedstock is one or more α-alkenes, one or more linear alkenes having a disubstituted double bond, or a combination thereof.

59. The process of claim 56, wherein the Cₙ-alkene feedstock is one or more linear alkenes having a disubstituted double bond.

60. A process for producing an isomeric mixture of at least one x-Cₙ-alkene comprising,
a) contacting the Cₙ-alkene feedstock with an additional material selected from Rh, Ir, Ni, Pd, Pt, and solid acid catalysts to initially convert the Cₙ-alkene feedstock to an isomeric mixture of at least one z-Cₙ-alkene, wherein n is one or more integers selected from 8 to 40; and z defines the position of the alkene double bond in the n-carbon alkene chain wherein the average value of z is between 2 and n/2;
b) contacting the z-Cₙ-alkene with a heterogeneous catalyst system comprising a group IV metal oxide at a temperature and pressure conducive to positional isomerization of the double bond and recovering an isomeric mixture of at least one x-Cₙ-alkene;
wherein n is one or more integers selected from 8 to 40;
x defines the position of the alkene double bond in the n-carbon alkene chain wherein the average value of x is between 2 and n/2;
z defines the position of the alkene double bond in the Cₙ-alkene feedstock;
the average value of z is between 1 and n/2; and
the average value of x is greater than the average value of z.
